(19) United States
(12) Patent Application Publication
(10) Pub. No.: US 2019/0183442 A1
(43) Pub. Date: Jun. 20, 2019

(54) PORTABLE DIGITAL RADIOLOGICAL CASSETTE

(71) Applicant: TRIXELL, MOIRANS (FR)

(72) Inventors: Philippe LE BECHENNEC, MOIRANS (FR); Cédric TUAILLON, MOIRANS (FR)

(21) Appl. No.: 16/209,891

(22) Filed: Dec. 4, 2018

(30) Foreign Application Priority Data
Dec. 14, 2017 (FR) 1762131

(51) Int. Cl. A61B 6/00

(52) U.S. Cl. CPC A61B 6/4283; A61B 6/4405

(57) ABSTRACT

A method for packaging a housing having an essentially parallelepipedal form defined by main faces includes a first larger face and a second larger face and lateral faces, the lateral faces being separated in pairs by corners of the housing, the housing housing a digital detector of ionizing radiation in the form of a flat panel, the method comprising a main step of covering of the housing by several films comprising a first step of covering of the first larger face of the housing by a first film configured, positioned and oriented relative to the first larger face so as to include flaps extending beyond the first larger face and capable of being folded independently of one another to cover adjacent lateral faces.

### CROSS-REFERENCE TO RELATED APPLICATIONS

This application claims priority to foreign French patent application No. FR 1762131, filed on Dec. 14, 2017, the disclosure of which is incorporated by reference in its entirety.

### FIELD OF THE INVENTION

The invention relates to a portable radiological cassette intended to equip a digital radiological system. The cassette comprises a digital detector of ionizing radiation that makes it possible to provide an image that is a function of the received radiation.

### BACKGROUND

The radiological system further comprises a source of ionizing radiation, such as an X-ray tube for example, making it possible to generate an X radiation, and a base station comprising an information processing system that makes it possible to synchronize the X-ray tube and the detector and that also makes it possible to perform image processing operations such as presenting to the operator the image corrected of all the defects inherent to the detector and enhanced, for example, by outline enhancing processing operations. An object for which an X-ray image is to be obtained is placed between the source and the detector. Such a system can be used in many applications such as, for example, medical radiology and non-destructive inspection. The invention can also be implemented for other types of radiations to be detected, in particular gamma radiations.

In the past, the radiological systems were bulky and immobile. It was necessary to position the object relative to the system to obtain the desired image. With the emergence of solid-state detectors as for example described in the French patent application FR 2 605 166, the detector has become less bulky and it is possible to move the detector relative to an object that remains fixed. For medical radiology, digital detectors have been produced in the form of mobile cassettes that can now be placed in immediate proximity to a patient for whom an image is to be produced, when the state of health of the patient prevents his or her travel to a room reserved for radiology.

The portable cassette comprises a housing primarily ensuring the function of mechanical support and of protection of the detector from deformations when dropped. The housing has an essentially parallelepipedal form. The radiation to be detected passes through the housing through one of its larger faces.

The outer surface of the housing can be left untreated or else be painted. Leaving the outer surface untreated often provides an unsatisfactory aesthetic, and a poor suitability to cleaning which has to be frequent in the case of use of the cassette in medical radiology. Painting the outer surface of the housing makes it possible to improve the aesthetic but the robustness remains limited faced with mechanical abuses such as impacts, scratches or even abrasion due to the repeated handling of the cassette.

An alternative solution consists in covering the housing with a coating based on plastic films to ensure the tightness of the cassette to liquids and to dust.

This type of coating also enhances the mechanical resistance of the outer surface of the cassette to scratches and abrasion and makes it possible to withstand chemical abuses, in particular from cleaning and disinfection products. For the face exposed to the radiation, it exhibits a minimal and uniform attenuation of the radiation.

Moreover, the cassette, in its use in medical radiology, is required to be placed on trolleys and, more generally, on hospital furniture. The cassette has to have a friction coefficient that is sufficient to stop rapidly if it is placed with a certain speed and remain in place once placed, even under accidental stresses. This implies an adequate friction coefficient. Conversely, in the phases of use, the friction coefficient has to be low enough to allow the detector to be slid under a patient without excessive effort. A film is much more resistant against mechanical abuses than paint, has a minimum of roughnesses and of retention zones detrimental to its suitability for cleaning, and can, ultimately, allow for a renewal of the outer appearance of the cassette through replacement of the films. The replacement is in particular useful in the presence of scratches in which impurities can accumulate.

A solution disclosed in the French patent application bearing the publication number FR3000344 consists in successively covering each of the larger faces of the housing with a plastic film and of covering three lateral faces of the housing and of making these films overlap over three lateral faces of the housing making them mold to the surface of the housing by thermoforming at a temperature of the order of 110° C. The thermoforming is done in a vacuum so as to ensure the lamination of the film on the housing and avoid the formation of folds. The two films overlap on the lateral faces of the housing.

This method presents a certain number of drawbacks.

In the thermoforming phase, each film is stretched and it is therefore made thinner, particularly in the corners of the housing, at the point where the housing is subjected to strong mechanical stresses which embrittle it which provokes its premature ageing. The stretching of the films also provokes a thinning of the thickness of the glue on the perimeter of the larger faces which limits the robustness of the protection provided by the films. The temperature-stressing of the films tends to make them breakable which leads to their premature ageing. Moreover, the housing conventionally has aluminum parts to which the films are difficult to glue. In effect, the aluminum, which is a good thermal conductor, forms an obstacle to the hardening of the glue which locally remains at too low a temperature.

Since thermoforming requires a heating at high temperature, the customizing of the cassette by printing can be done only after the phase of gluing on the film which embrittles this customization which is subject to significant stresses. Moreover, this method is costly and lengthy, it requires heavy equipment, particularly for making the vacuum.

### SUMMARY OF THE INVENTION

One aim of the invention is to limit at least one of the abovementioned drawbacks.

To this end, the subject of the invention is a method for packaging a housing having an essentially parallelepipedal form defined by main faces including a first larger face and a second larger face and lateral faces, the lateral faces being separated in pairs by corners of the housing, the housing housing a digital detector of ionizing radiation in the form of a flat panel, the method comprising a main step of covering of the housing by several films comprising a first step of covering of the first larger face of the housing by a first film configured, positioned and oriented relative to the first larger face so as to include flaps extending beyond the first larger face and capable of being folded independently of one another to cover adjacent lateral faces.

Advantageously, the first film includes at least three flaps extending beyond the first large face and capable of being folded independently of one another to cover at least three of the lateral faces of the housing.

Advantageously, the method comprises a step of folding of the flaps such that the flaps cover adjacent lateral faces of the housing.

Advantageously, the flaps extend beyond adjacent lateral faces, the method comprising a step of folding of the flaps such that a remainder of the flaps covers the second larger face.

Advantageously, the main covering step comprises a step of covering of the second larger face by a second film, the second film covering only the second larger face.

Advantageously, the main covering step is performed such that the two films do not overlap, the method comprising a step of sealing of an interstice between the first film and the second film by a seal.

Advantageously, the main covering step comprises a step of cutting and of removal of edges of the remainders of the flaps covering the second larger face along a cutting line of predetermined position relative to the second larger face and a step of cutting and of removal of an edge of the second film along a closed second cutting line of predetermined position and form relative to the first cutting line.

Advantageously, the method comprises a step of covering of the corners of the housing by corner protections.

Advantageously, the method comprises a step of cutting and of removal of edges of the first film surrounding corners of the housing so as to leave free parts of the housing including and surrounding corners of the housing, the corner protections being arranged so as to cover the free parts without covering the first film.

Advantageously, the thicknesses of each corner protection and of each film are chosen such that, when the cassette is placed on a flat support, only the corner protections are in contact with the flat support.

Advantageously, at least one film comprises a plastic film, an assembly layer making it possible to make the first film adhere to the housing and a printing layer interposed between a plastic film and the assembly layer.

The invention relates also to a portable radiological cassette comprising a housing having an essentially parallelepipedal form defined by main faces including a first larger face and a second larger face and lateral faces, the lateral faces being separated in pairs by corners of the housing, the housing housing a digital detector of ionizing radiation in the form of a flat panel, the housing being covered by several films comprising a first film covering the first large face, the first film including flaps extending beyond the first larger face and folded independently of one another to cover adjacent lateral faces.

Advantageously, a remainder of each flap covers the second larger face.

Advantageously, the cassette comprises a second film covering only the second larger face.

Advantageously, the radiological cassette includes a seal sealing an interstice between the first film and the second film.

Advantageously, the radiological cassette includes corner protections covering the corners.

Advantageously, the corner protections do not cover the films.

Advantageously, the thickness of each corner protection and of each film is chosen such that, when the cassette is placed on a flat support, only the corner protections are in contact with the flat support.

Advantageously, at least one film comprises a plastic film, an assembly layer making it possible to make the first film adhere to the housing and a printing layer interposed between a plastic film and the assembly layer.

### BRIEF DESCRIPTION OF THE DRAWINGS

The invention will be better understood and other advantages will emerge on reading the detailed description of an embodiment given by way of example, the description being illustrated by the attached drawing in which:

FIG. 1 represents a radiological system implementing the invention;

FIG. 2 represents, in perspective, a housing of a cassette according to the invention;

FIGS. 3a to 3k represent a housing and then the intermediate products obtained after the different steps of the method according to the invention, and the cassette seen from above facing a first larger face of the parallelepiped defined by the housing (FIGS. 3a, 3b) and facing the second larger face of the parallelepiped defined by the housing (FIGS. 3c to 3k);

FIG. 4 schematically represents a cross-sectional view of the intermediate product of FIG. 3i along the cutting plane AA,

FIG. 5 schematically represents a cross-sectional view of a first film F1,

FIG. 6 schematically represents a cross-sectional view of the cassette along the cutting plane BB of FIG. 3k.

In the interests of clarity, the same elements will bear the same references in the different figures.

### DETAILED DESCRIPTION

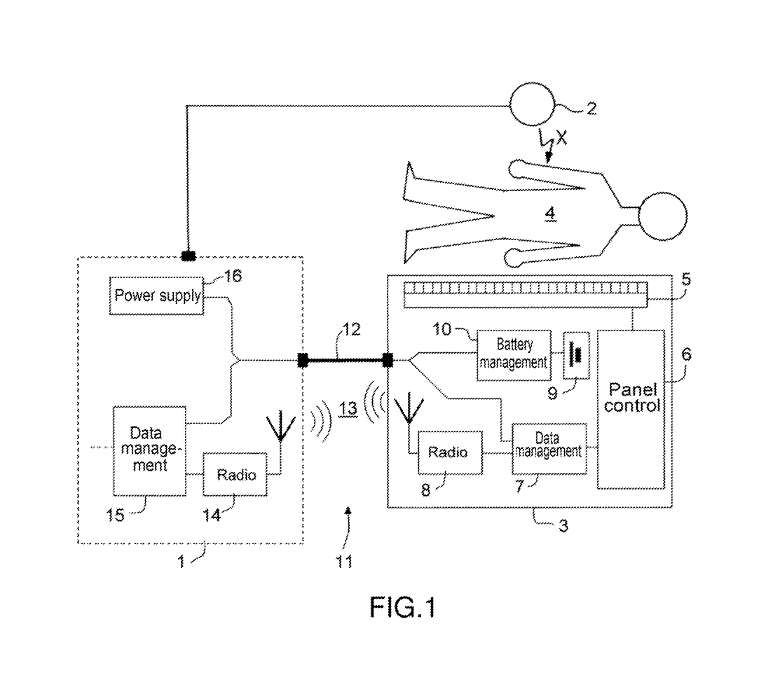

FIG. 1 represents a radiological system intended for a medical use. The system comprises a fixed base station 1, an X-radiation generator 2 and a radiation detector in the form of a portable cassette 3. The cassette makes it possible to obtain an image of a patient 4 passed through by the X-radiation from the generator 2. The cassette 3 comprises a digital detector produced in the form of a flat panel 5 linked to a control module 6 that makes it possible to read the image obtained by the flat panel 5 and to digitize it through an analogue/digital converter. The mobile cassette 3 also comprises a data management module 7, a radio module 8, a battery 9 and a battery management module 10.

The base station comprises a radio module 14, a data management module 15 and a power supply 16.

Means of communication 11 between the cassette 3 and the base station 1 make it possible to transfer data such as the image between the cassette 3 and the base station 1. The data can circulate either from the base station 1 towards the cassette 3, or from the cassette 3 towards the base station 1. Towards the cassette 3, this concerns, for example, command information from the flat panel 5, and to the base station 1, the data comprises, for example, the images produced by the flat panel 5.

The communication means can comprise a removable wired link 12 and/or a wireless link 13. The two links 12 and 13 are both capable of transferring the data. The two radio modules 8 and 14 make it possible to exchange the data between the base station 1 and the cassette 3. The data management module 7 of the cassette 3 makes it possible to switch the data received or originating from the control module 6 to one of the links 12 or 13. Likewise, in the base station 1, the data management module 15 makes it possible to switch the data received or originating from one of the links 12 or 13. The power supply 16 provides the electrical energy necessary to the operation of the different modules of the base station 1 and of the cassette 3.

The powering of the cassette 3 is done via the wired link 12 or the battery 9. Advantageously, the system comprises means for recharging the battery 9. More specifically, the battery management module 10 measures the charge of the battery 9 and provokes the recharging thereof in case of need.

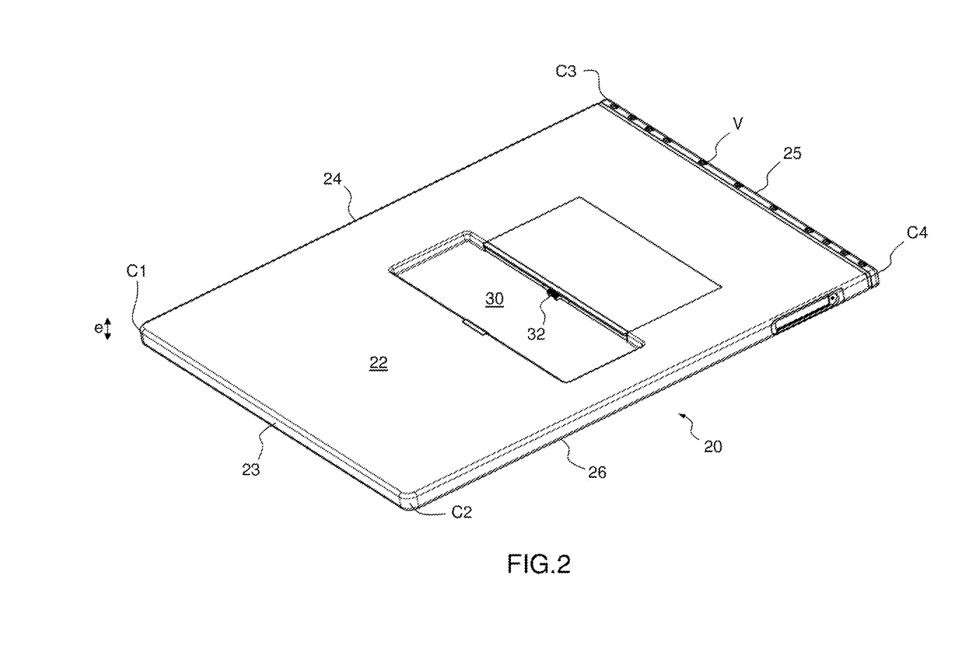

FIG. 2 represents a housing 20 of the radiological cassette 3 according to the invention. The housing 20 has an essentially parallelepipedal form. The digital detector 5, the control module 6, the data management module 7, the radio module 8 and the battery management module 10 are arranged inside the housing 20. These four modules are arranged on an electronic circuit board. The modules are given only by way of example. They are not mandatory for the implementation of the invention. The battery 9 is arranged outside of the housing 20 in order to facilitate a possible replacement.

The housing 20 has six main faces 21 to 26 delimiting the substantially parallelepipedal form. The six faces are parallel in pairs. The main larger faces 21, 22 are linked to one another by the corners of the housing C1, C2, C3, C4 separated in pairs by the lateral faces 23 to 26. The detector 5, in the form of a flat panel, has a radiation detection surface close to that of the two larger faces 21 and 22 parallel to one another. The other faces 23 to 26 are the smaller faces of the housing 20. In the embodiment of the figures, the parallelepiped is essentially rectangular. In a variant, the faces of the parallelepiped are not rectangular.

The battery 10 is housed within the parallelepipedal volume formed by the housing 20. The battery 10 is housed on the outside of the housing 20 in a recess 30 produced in the face 22. The recess 30 forms a hollow in the second larger face 22, the hollow complementing the form of the battery 9 so that, once put in place, the battery 9 does not extend beyond the face 22.

The digital detector 5 is arranged inside the housing 20 on the side of the face 21. The face 21 is substantially flat.

The cassette 3 comprises at least one connector 32 arranged through a wall of the housing 20.

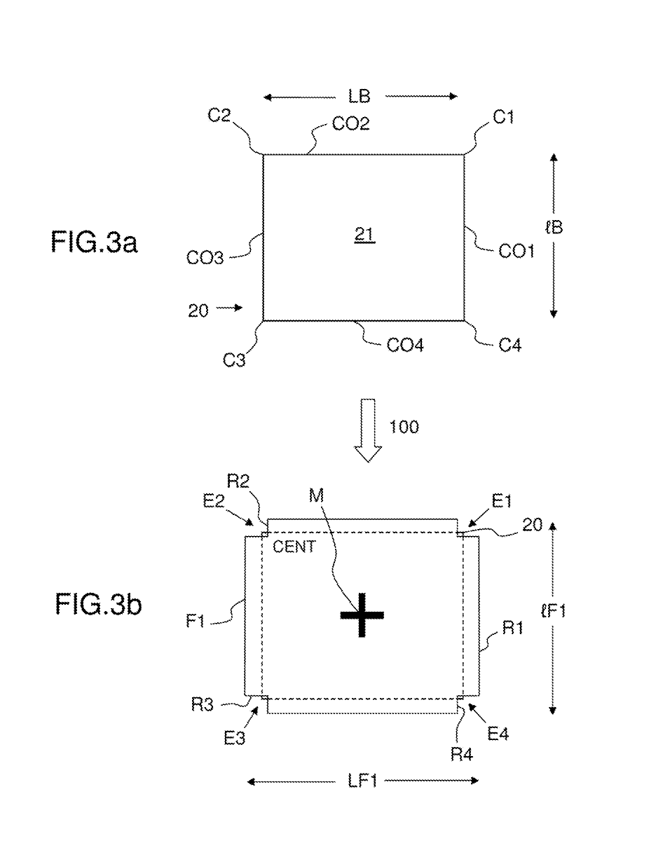

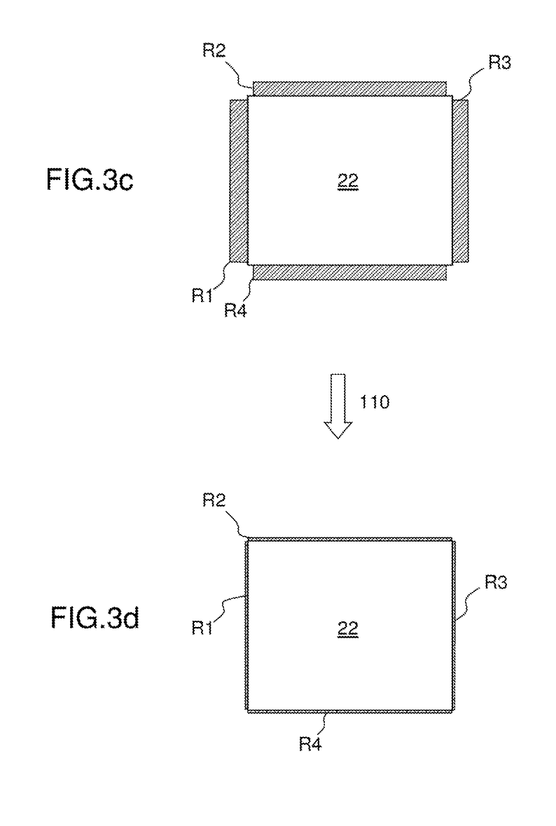

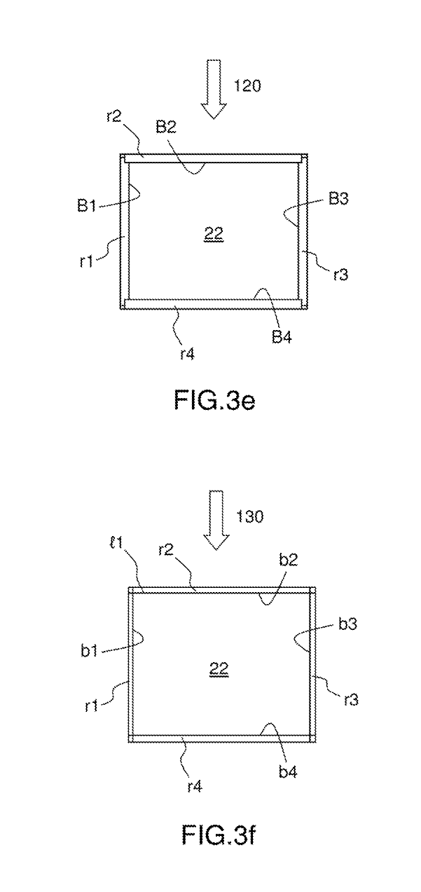

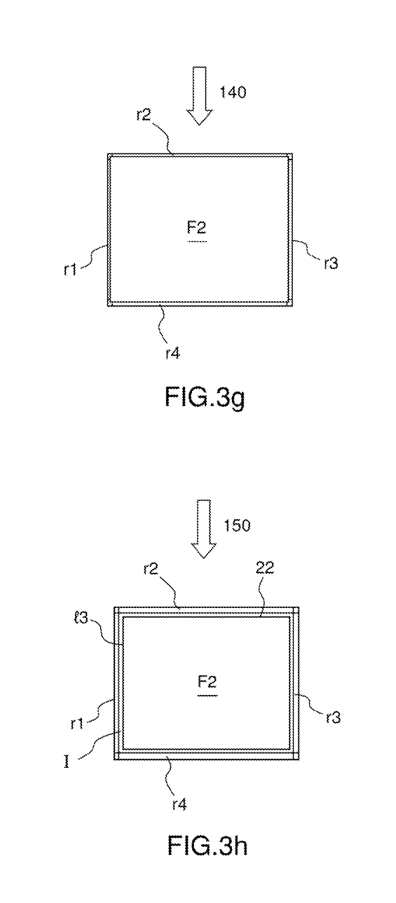

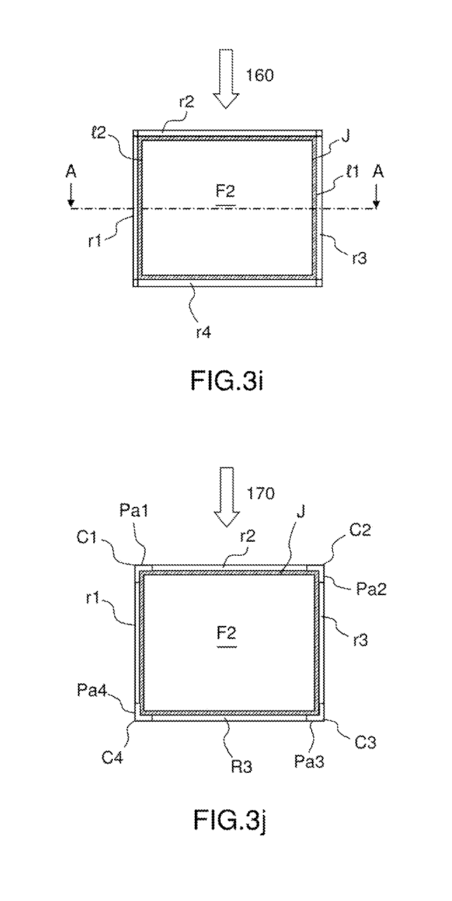

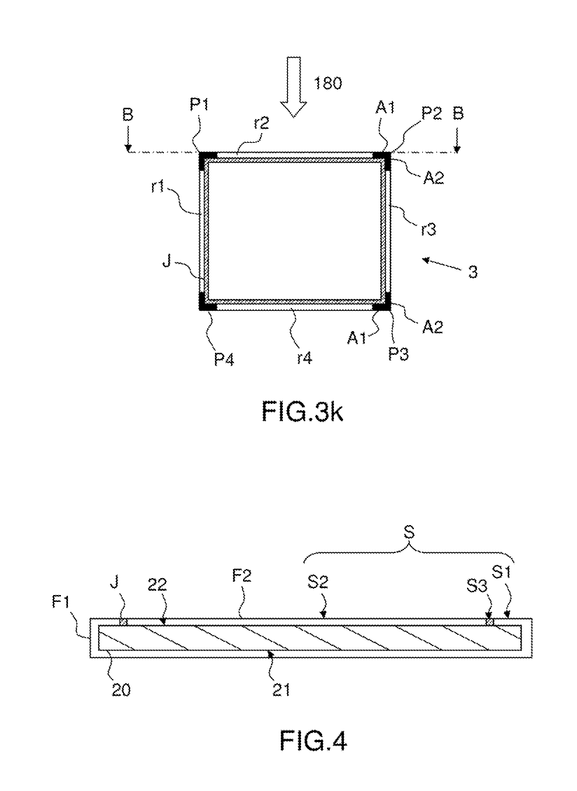

FIGS. 3a to 3k represent the initial product (FIG. 3a) then the intermediate products obtained in the various steps (represented by arrows) of the method according to the invention so as to obtain the radiological cassette 3 according to the invention.

The packaging method consists in covering the housing with a coating laminated on the housing.

In these drawings, for greater clarity, the housing 20 is represented by a perfect rectangular parallelepiped without any recess.

The packaging method according to the invention comprises a main covering step for covering the housing with several films.

This main covering step comprises a first step 100 of covering of the first larger face 21 by a first film F1. In the method according to the invention, in a step of covering of a main face of the parallelepiped by a film, the film is made to adhere to the main face and the film is laminated on the main face over all the part of its surface covering the main face.

The first film F1 is configured but also positioned and oriented relative to the first large face 21 so as to include flaps R1, R2, R3, R4 extending beyond the first large face 21 beyond the four sides CO1, CO2, CO3, CO4 of the first large face 21 as is represented in FIGS. 3b and 3c representing the housing 20 covered with the first film F1 at the end of the covering step 100, facing the first large face 21 (FIG. 3b) and facing the second face 22 (FIG. 3c). Configuration of the film should be understood to mean in particular the dimensions and form thereof.

As can be seen in FIGS. 3b and 3c, each flap R1, R2, R3 and R4 extends beyond the first large face 21 and is capable of being folded independently of the other flaps to cover one of the lateral faces 23 to 26 of the housing 20.

In other words, once the first film F1 covers the first face 21, it is fixed and attached to this first face 21 in the position defined above and represented in FIG. 1, it is possible to fold each flap R1, R2, R3 and, respectively, R4 along one of the sides CO1, CO2, CO3, and, respectively, CO4 of the large face 21 without folding the other flaps. Thus, the adjacent flaps are separated from one another so that each of these flaps can be folded along one sides CO1, CO2, CO3, or, respectively, CO4 of the housing separating the first larger face 21 from one of the lateral faces 23, 24, 25 or 26. The two adjacent flaps are folded along edges having different directions so as to cover adjacent lateral faces while being laminated on the housing over all their surfaces. This method makes it possible to cover the different lateral faces 23 to 26 of the housing 20 without thermoforming and therefore avoid the drawbacks associated with thermoforming. It is possible to cover each lateral face 23 to 26 of the housing only by folding while ensuring a continuous lamination of the film on the housing.

Thus, the packaging method according to the invention makes it possible to guarantee a constant film thickness over all its surface, to guarantee an adhesion on the aluminum that is significantly superior to that of a thermoformed film, to guarantee a constant thickness of adhesive over all its surface glued to the housing, and not to subject the film to premature ageings. It therefore makes it possible to increase the robustness of the detector with respect to the environment, and to facilitate and improve the cleaning. It also makes it possible to reduce the cassette manufacturing costs and time. Placement in a vacuum is not necessary to laminate the film, over all its surface, on the housing. The method can include a step of heating to a moderate temperature lying between 70° C. and 90° C. during which the main covering step is performed, in order to favor the folding of the film and avoid the subsequent shrinkage problems.

The absence of thermoforming that naturally provokes an uncontrolled deformation of the printing (pattern, text) in the stretching of the plastic film makes it possible to incorporate a printing layer in the film and therefore to customize the cassette at the latest possible moment while making it possible to ensure its protection as will be seen hereinbelow.

The overall length LF1 of the first film F1 is greater than the length LB of the first face 21 and the overall width IF1 of the first film is greater than the width IB of the first face 21. Moreover, the film includes, on its periphery, four parts intended to form the four flaps R1 to R4. These four parts are separated by cutouts E1, E2, E3, E4 referenced in FIG. 4 representing the first film F1 alone and making it possible to fold the flaps R1 to R4 independently of one another. The four flaps R1 to R4 surround a central part CENT, delimited by dotted lines in FIG. 3b, covering the first face 21, relative to which the flaps R1 to R4 can be folded independently of one another.

Thus, the first film F1 has, in the nonlimiting example of FIG. 3b, essentially a rectangular form without its vertices. The first film comprises cutouts E1 to E4 removing the vertices of the rectangle substantially formed by the first film.

In the embodiment of the figures, the cutouts E1 to E4 are substantially rectangular. This form is not limiting, it is for example possible to consider notches in the form of quarter circles or substantially linear.

In the nonlimiting embodiment of the figures, the central part CENT of the first film F1 is intended to cover substantially all the first face without covering its vertices which are each located substantially at the intersection of two adjacent sides CO1, CO2, CO3, CO4. As a variant, the central part CENT of the first film F1 is intended to cover all the first face 21.

The step 100 of covering of the first large face 21 by the first film F1 is followed by a step 110 of covering of the four small faces 23 to 26 by the first film F1 whose flaps are substantially parallel to the small faces 23 to 26 at right angles to the plane of FIG. 3d. More specifically, the step 100 of covering of the first large face 21 by the first film F1 is followed by a step of folding of each of the flaps R1 to R4 such that it covers one of the four small faces 23 to 26. Each flap 23 advantageously covers just one of the four small faces of the housing of the cassette 3.

The folding of each flap R1 to R4 is performed along a side CO1 or CO4 of the first large face 21 such that the first film molds to the form of this side.

Advantageously, each flap R1 to R4 extends beyond the small face 23 to 26 that it covers, such that it can cover the second larger face 22. In other words, the width of each flap R1 to R4 is greater than the thickness e of the housing 20 taken in the direction at right angles to the two larger faces.

Advantageously, the step 110 of covering of the four small faces 23 to 26 by the first film F1 is followed by a step 120 of folding of a remainder r1 to r4, of each flap R1 to R4, extending beyond the small face 23 to 26 covered by the flap R1 to R4 such that the remainder r1 to r4 of the flap r1 to r4 extends over the second large face 22 as can be seen in FIG. 3e. It should be noted that it would be difficult to have the first film covering the first larger face 21 extend over the second larger face 22 by thermoforming without provoking a thinning of the film possibly going as far holing it.

In the nonlimiting example of the figures, the method comprises a step 130, of cutting and of removal of the edges B1, B2, B3, B4 of the remainders of the flaps r1, r2, r3, r4 covering the second large face 22, along a closed cutting line l1, represented in FIG. 3f, of predetermined position relative to the second large face 22. This cutting line l1 is advantageously substantially rectangular, as represented in FIG. 3f. This step reduces the size of the remainders of the flaps r1, r2, r3, r4 and leaves free new edges b1, b2, b3, b4 of the remainders of the flaps.

The step 130 is followed by a step 140 of covering of the second large face 22 with the second film F2, that can be seen in FIG. 3g, such that the second film F2 covers a central part of the second large face 22 and extends over the remainders of the flaps so as to totally cover the new edges b1, b2, b3, b4 of the remainders of the flaps r1, r2, r3, r4.

Advantageously, each of the small faces is covered with a single film taken from among the films F1 and F2.

Advantageously, the second film F2 covers only the second face 22 of the cassette 3 and the join between the films F1 and F2 is located on the second large face 22 which facilitates the industrial performance of the method relative to a joining of the films on the sides, that is to say on the small faces 23 to 26 of the housing, and which favors the robustness of the coating to external abuses, the housing being less stressed on its large faces.

As a variant, the two films F1 and F2 overlap on the small faces or sides of the housing 20.

The step 140 is followed by a step 150 of cutting of the edge of the second film F2 along a second closed cutting line l2, visible in FIG. 3h, and of removal of this edge of predetermined position and form relative to the first cutting line l1 such that the two films do not overlap. The absence of overlapping of the films makes it possible to limit the risks of tearing of the packaging. This method makes it possible to control the spacing between the two films. An interstice I in the form of a closed loop completely surrounding the second film F2.

Advantageously, the cutting line l2 has substantially the same form, substantially in the same proportions, as the line l1 and one and the same center. The line l2 delimits a smaller zone than the line L1 in order to avoid any overlapping between the films F1 and F2. Thus, the spacing between the two films F1 and F2 is substantially fixed over all the perimeter of the cutting line l2.

Advantageously, the size of the cutting lines l1 and l2 is such that the spacing between the lines l1 and l2 in the plane of the face 22 is substantially a gap making it possible to guarantee an absence of overlap between the two films. In other words, the films are cut edge-to-edge.

The cutting steps are advantageously laser cutting steps, the laser being displaced by a rig relative to a support along one and the same fixed closed line relative to the support, during the two cutting steps, the housing occupying one and the same position and one and the same orientation relative to the support during these two steps. Thus, the distance between the lines l1 and l2 is substantially equal to the diameter of the laser spot on the face of the housing. The diameter of the spot is of the order of 100 micrometers. This method exhibits very high cutting and alignment accuracies of the order of 10 micrometers.

As a variant, the cutting steps are mechanical cutting steps.

Advantageously, the method comprises a sealing step 160 consisting in applying a seal J, visible in FIG. 3i, to fill the interstice I formed between the first film F1 and the second film F2, in the step 140. This step makes it possible to ensure the seal-tightness of the coating R while limiting the presence of zones of accumulation of dust or of liquids and therefore of germs or of bacteria which facilitates the cleaning of the cassette. This configuration guarantees a good mechanical resistance of the enclosure because it avoids the overlapping of the films and consequently the presence of an edge of one of the films on the other film favoring the tearing of the film in the form of excess thickness.

The seal J is deposited over all the perimeter of the interstice I formed between the two films F1 and F2.

In the embodiment of the figures, the seal J totally fills the interstice I. The seal J is flush with the free surfaces of the first film F1 and of the second film F2. A continuous substantially flat surface S is formed by the free surface S3 of the seal and by the free surfaces S1 and S2 of the films F1 and F2 in the vicinity of the seal as represented in FIG. 4 representing a cross section of the cassette along a cutting plane A-A. That also makes it possible to limit the presence of zones of buildup of dust or of liquids and therefore of germs or of bacteria and improve the mechanical resistance of the enclosure.

As a variant, the seal J partially fills the interstice I.

The films F1 and F2 advantageously have approximately the same thickness.

As a variant, the method comprises a step of fusing of the edges of the two films F1 and F2 so as to ensure a continuity of material between the two films and thus limit the zones of buildup of dust and of bacteria.

Advantageously, in this case, the two films overlap before the fusing step. For example, the method does not include the cutting step 130 and/or the cutting step 150. As a variant, the method includes the cutting steps 130 and 150.

FIG. 5 shows an exemplary embodiment of the first film F1. The structure of the second film F2 is not represented but is advantageously similar to that of the film F1.

The film F1 is multilayer and includes a layer of plastic film 70 and a layer of assembling material 75 making it possible to make the plastic film 70 adhere to the housing 20. In other words, the film F1 and the housing 20 are assembled by direct adhesion. The film F1 is then attached to the housing 20. The films are positioned, in the covering steps, such that the layer of assembly material or adhesive material 75 is interposed between the plastic film 70 and the face covered by the film so as to make the film F1 and the housing 20 adhere.

The plastic film has a thickness of between 150 μm and 700 μm; for example 200 μm and the layer of assembly material has a thickness of between 50 μm and 300 μm, for example 130 μm.

Advantageously, the layer of assembly material extends over all the surface of the film.

As a variant, the film F1 comprises a layer of double-sided adhesive tape comprising a tape coated with a layer of adhesive material on each of its two opposite faces of which one face is fixed to the plastic film 70 and of which a second face is intended to face the housing 20. Advantageously, the tape is opaque to the visible spectrum and transparent to the X rays. Thus, the outer color of the housing is that of the tape if the adhesive is transparent.

Advantageously, the assembly material 75 is a soft solid which makes the plastic film 70 adhere to the housing 20 by deformation of the adhesive under the effect of a pressure. This solution offers the advantage of not necessarily requiring any heating and of being rapid.

The method can include a step of heating to a temperature lying within a temperature range in which the plastic film can be deformed in a stable manner. This temperature for example lies between 70° C. and 90° C. That makes it possible to obtain a lamination of the plastic film 70 without defects (air bubbles) and without the risk of lifting over time.

The heating step is, for example, implemented during the step 110 to favor the conformation of the film and the lamination of the film on the sides.

As a variant, the assembly material 75 is a glue, for example a thermosetting resin glue. It makes the film and the housing adhere by hardening, for example by crosslinking of the glue.

Alternatively, the method comprises a step of application of an adhesive material, for example a glue, on the faces of the housing 20 preceding the steps of covering of the housing by the films. This configuration makes the control of the thickness of the cassette over all its surface more difficult. Now, the aim is to obtain a cassette that has the smoothest possible surface and a thickness that is substantially constant over all its surface.

Advantageously, at least one film comprises at least one printing layer 76 arranged between the plastic film 70 and the layer of assembly material 75 which makes it possible to ensure a protection of the printing layer by the plastic film and thus improve the robustness of the printing. This printing layer is a layer of ink, deposited for example by screen printing. The printing is intended to customize the housing. It can comprise at least one text and/or at least one pattern such as, for example, a cross in the example of the figures. Advantageously, the plastic film 70 is transparent to at least a part of the visible spectrum so that the printing is visible when the plastic film is glued onto the housing. This configuration makes it possible to consider multiple possibilities of customization of the housing with different patterns and colors on the different faces of the housing without any face-by-face customization operation after the packaging of the housing.

The overall thickness of the printing layer lies between 10 μm and 50 μm. It forms, for example, a thickness of 10 μm.

The film F2 can be interrupted at the recess 30, particularly at the connector 32 so as not to cover the connector 32. Furthermore, there is no need to cover the bottom of the recess 30, the bottom parallel to the face 21. Indeed, this bottom is not subjected to the same mechanical abuses as the outer faces 21 and 22 because it is protected by the battery 9. The bottom of the recess 30 can be left untreated or covered with a paint.

Generally, at least one film can comprise, before the step of covering of a face of the housing by the film, at least one opening, for example delimited by a closed curve intended to be arranged facing zones of the housing intended to be left free by the packaging method, for example by the coating. This feature makes it possible to avoid a cutting of the film after the covering of the housing to free these zones. The presence of these prior openings is not allowable in a vacuum-forming. Indeed, vacuum-forming requires a perfect seal-tightness. In the case of thermoforming, the plastic film sheet participates in the sealing so, if it is perforated (hole or opening), the vacuum cannot be achieved. It is therefore not possible to make prior cuts in the film.

The films F1 and F2 advantageously mask fixing means of screw or nut type V. This masking makes it possible to avoid any build-up of impurities in the fixing means in the use of the cassette 3. The cleaning of the cassette 3 is facilitated thereby. The masking also makes it possible to limit the opening of the cassette 3 by an unauthorized person.

The method possibly comprises, as can be seen in FIG. 3j, a step 170 of cutting and removal of the edges of the first film surrounding the corners of the housing so as to leave free parts Pa1 to Pa4 of the housing including and surrounding the corners C1, C2, C3, C4.

The method then includes, as can be seen in FIG. 3k, a step of covering 180 of the corners C1, C2, C3, C4 and more specifically of the parts Pa1 to Pa4 of the housing 20 by corner protections P1, P2, P3, P4 so as to obtain the radiological cassette 3. The corner protections make it possible to mechanically protect the corners. They have a reinforcing function.

The housing 20 of the cassette 3 is covered with a coating R comprising the films F1 and F2, the corner protections P1 to P4 and the seal J.

Advantageously, the corner protections cover the free parts Pa1 to Pa4 without covering the plastic films. That makes it possible to limit the total thickness of the cassette which is standardized and to limit the risks of catching of the corner protections and thus limiting the risks of injuring the patients.

The method can include a step of deposition of a seal to fill a space between at least one plastic film and a corner protection.

Each corner protection P1, P2, P3, P4 is configured and arranged facing a corner C1, C2, C3 or C4 of the housing 20 so as to cover the corresponding corner C1, C2, C3 or C4. Each corner protection also partially covers the faces F1 and F2.

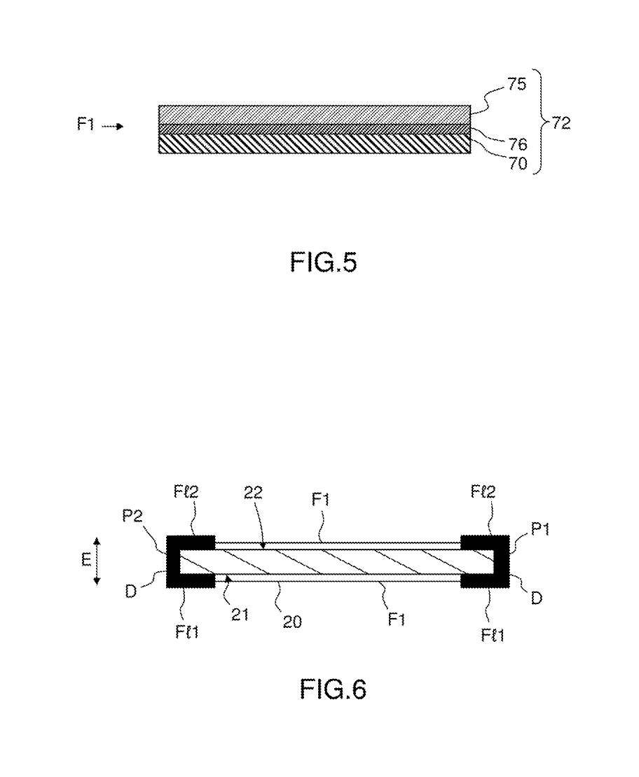

In the example represented in FIGS. 3k and 6, the corner protection comprises two wings A1, A2 linked to one another so as to define an angle of 90°. The two wings each comprise a U-shaped section. In other words, they each have two flanks Fl1, Fl2 linked by a back D. Each wing A1, A2 comprises a flank Fl1 covering a part of the first large face 21 situated in proximity to a corner and another flank Fl2 covering a part of the second large face 22 situated in proximity to the same corner, the backs D covering the parts of the small faces linked by the corner. Each back D covers only one of the small faces. The two wings of each corner protection meet facing the corner that they cover.

Each corner protection comprises, for example, a metal layer. That confers a more solid mechanical protection on the corners of the housing which are the most stressed parts and that facilitates the production of the corner protection which has a complex form and a small thickness. As a variant, the corner protections can for example be a layer of plastic material. They can for example have the same structure and composition as the films F1 and F2. The corner protection advantageously comprises a layer of assembly material.

Advantageously, each corner protection P1, P2, P3, P4 has a thickness defined such that, when the plate is placed on a flat support, it rests on the flat support only by the corner protections. That makes it possible to limit the exposure of the films to the mechanical abuses (frictions) and/or chemical abuses (products) by avoiding the contact of the films with the support. The thickness of the corner protections lies between 300 μm and 400 μm. This thickness is the thickness of the flanks Fl1 and Fl2.

The films F1 and F2 typically have a thickness of between 200 μm and 1000 μm. It is for example 330 μm.

In the step of covering of the corners by the corner protections, the corner protections are fixed to the housing. The corner protections can be multilayer and comprise an assembly layer as described previously or else the method can include a step of application of an assembly layer on the corner prior to the step of covering of a corner by the housing.

The face 21 is intended to be passed through by the ionizing radiation to be detected. It is transparent to the radiation so as to allow the detector to receive the radiation. The film F1 covering this face or at least its central part, is also transparent to the ionizing radiation to be detected.

The housing 20 can comprise an enclosure produced in a single-piece mechanical part forming five first faces of the essentially parallelepipedal form including the two larger faces, and a plug to form a sixth face of the essentially parallelepipedal form. In this case, the first film can comprise only three flaps that can be folded independently of the other flaps to cover one of the lateral faces of the housing 20. More generally, to avoid thermoforming, the method comprises a step of covering of a large face of the housing by a first film so as to comprise at least two flaps that can be folded independently of one another to cover two adjacent lateral faces of the housing 20.

In the embodiment of FIG. 2, the first face 21 is substantially flat and the second face 22 includes the recess. As a variant, the second face 22 is substantially flat and the first face includes the recess 30.

In the past, medical radiology used silvered films which were handled in cassettes. The standard ISO 4090 has defined the dimensions of cassettes enclosing the silvered films. The thickness E of the cassettes defined by the standard is between 13 and 16 mm. Advantageously, the cassette 3 according to the invention meets, with respect to its dimensions, the requirements of the standard ISO 4090. More particularly, the overall thickness of the cassette 3 is less than 16 mm. This makes it possible to use silvered cassette storage means for a digital cassette 3 according to the invention.

1. A method for packaging a housing having an essentially parallelepipedal form defined by main faces including a first larger face and a second larger face and lateral faces, the lateral faces being separated in pairs by corners of the housing, the housing housing a digital detector of ionizing radiation in the form of a flat panel, the method comprising a main step of covering of the housing by several films comprising a first step of covering of the first larger face of the housing by a first film configured, positioned and oriented relative to the first larger face so as to include flaps extending beyond the first larger face and capable of being folded independently of one another to cover adjacent lateral faces.

2. The method according to claim 1, in which the first film includes at least three flaps extending beyond the first large face and capable of being folded independently of one another to cover at least three of the lateral faces of the housing.

3. The method according to claim 1, comprising a step of folding of the flaps such that the flaps cover adjacent lateral faces of the housing.

4. The method according to claim 3, in which the flaps extend beyond adjacent lateral faces, the method comprising a step of folding of the flaps such that a remainder of the flaps covers the second larger face.

5. The method according to claim 1, in which the main covering step comprises a step of covering of the second larger face by a second film, the second film covering only the second larger face.

6. The method according to claim 5, in which the main covering step is performed such that the two films do not overlap, the method comprising a step of sealing of an interstice between the first film and the second film by a seal.

7. The method according to claim 6, in which the main covering step comprises a step of cutting and of removal of the edges of the remainders of the flaps covering the second larger face along a cutting line of predetermined position relative to the second larger face and a step of cutting and of removal of an edge of the second film along a closed second cutting line of predetermined position and form relative to the first cutting line.

8. The method according to claim 1, comprising a step of covering of the corners of the housing by corner protections.

9. The method according to claim 8, comprising a step of cutting and of removal of edges of the first film surrounding corners of the housing so as to leave free parts of the housing including and surrounding corners of the housing, the corner protections being arranged so as to cover the free parts without covering the first film.

10. The method according to claim 8, in which the thicknesses of each corner protection and of each film are chosen such that, when the cassette is placed on a flat support, only the corner protections are in contact with the flat support.

11. The method according to claim 1, in which at least one film comprises a plastic film, an assembling layer making it possible to make the first film adhere to the housing and a printing layer interposed between a plastic film and the assembly layer.

12. A portable radiological cassette comprising a housing having an essentially parallelepipedal form defined by main faces including a first larger face and a second larger face and lateral faces, the lateral faces being separated in pairs by corners of the housing, the housing housing a digital detector of ionizing radiation in the form of a flat panel, the housing being covered by several films comprising a first film covering the first large face and including flaps extending beyond the first larger face and folded independently of one another to cover adjacent lateral faces.

13. The portable radiological cassette according to claim 12, in which a remainder of each flap covers the second larger face.

14. The portable radiological cassette according to claim 13, comprising a second film covering only the second larger face.

15. The portable radiological cassette according to claim 14, including a seal sealing an interstice between the first film and the second film.

16. The portable radiological cassette according to claim 15, including corner protections covering the corners.

17. The portable radiological cassette according to claim 16, in which the corner protections do not cover the films.

18. The portable radiological cassette according to claim 17, in which the thickness of each corner protection and of each film is chosen such that, when the cassette is placed on a flat support, only the corner protections are in contact with the flat support.

19. The portable radiological cassette according to claim 12, in which at least one film comprises a plastic film, an assembly layer making it possible to make the first film adhere to the housing and a printing layer interposed between a plastic film and the assembly layer.
